(19) United States
(12) Patent Application Publication
(10) Pub. No.: US 2023/0125143 A1
(43) Pub. Date: Apr. 27, 2023

(54) SINGLE USE URETEROSCOPE WITH INTEGRATED SUCTION CATHETER

(71) Applicant: GYRUS ACMI, INC. D/B/A OLYMPUS SURGICAL TECHNOLOGIES AMERICA, Westborough, MA (US)

(72) Inventor: Jeffrey M. Schmitt, Bolton, MA (US)

(21) Appl. No.: 18/047,475

(22) Filed: Oct. 18, 2022

Related U.S. Application Data
(60) Provisional application No. 63/262,889, filed on Oct. 22, 2021.

(51) Int. Cl. A61B 1/018; A61B 1/00; A61B 1/005; A61B 1/307

(52) U.S. Cl. CPC A61B 1/018; A61B 1/00094; A61B 1/0052; A61B 1/00066; A61B 1/307

(57) ABSTRACT

A multi-channel endoscope can include a control section and an insertion end. The endoscope can include a user control mechanism. The control section can include at least one working channel and a channel within the control section. The working channel can accommodate at least one medical instrument which can fracture a target object into fragments. A catheter, such as a fluidic catheter, can extend from and retract to an originating proximal position at the insertion end of the endoscope. A vacuum pressure source or an irrigation source can be coupled to a proximal end of the channel. The user control mechanism can control the position of the catheter with respect to the endoscope and can be used to operate the vacuum pressure source or irrigation source.

### CLAIM OF PRIORITY

This application claims the benefit of priority of U.S. Provisional Patent Application Ser. No. 63/262,889, filed Oct. 22, 2021, which is hereby incorporated by reference in its entirety.

### BACKGROUND

A surgical device can provide access to surgical sites in a human body through a body opening, cavity, or tract. In certain urological procedures an opening can be dilated to accommodate one of several other medical devices. Instrumentation can be used such as to help mitigate the presence of stones (calculi) in the urinary tract. Endoscopic or ureteroscopic devices can have an articulation component, which allows the user to view, modify, repair, or otherwise interact with cavities and lumens within a human body. In some situations, a medical professional needs to remove fluid or other debris such as kidney stone fragments during procedures in which an endoscope or ureteroscope is used. For example, during a lithotripsy procedure, kidney stones may be broken down to small fragments and then the small fragments need to be removed from the ureter. In some cases, suction can be applied within the ureter to remove the kidney stone fragments. In another example, a flexible fiber with a basket-like component can be inserted into the ureter. The basket-like component can be used to grasp the kidney stone or kidney stone fragments and then remove the kidney stone or kidney stone fragments from the body. In another example, a small incision can be made in a body near the kidney and instruments can be inserted in the incision and then kidney. In this example, the medical professional can then remove the kidney stone or fragments of a kidney stone with a suction mechanism or other implements.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of an endoscope in accordance with at least one example of the present disclosure.

FIG. 2 illustrates an example of internal components within a control section of an endoscope in accordance with at least one example of the present disclosure.

FIG. 3 illustrates an example of an endoscope in accordance with at least one example of the present disclosure.

FIG. 4 illustrates an example of an insertion section of an endoscope in accordance with at least one example of the present disclosure.

FIG. 5 illustrates an example of a connector section of an endoscope in accordance with at least one example of the present disclosure.

FIGS. 6A and 6B illustrate examples of an insertion section of an endoscope in accordance with at least one example of the present disclosure.

FIG. 7A-7C illustrate examples of expandable suction catheters accordance with at least one example of the present disclosure.

FIGS. 8A and 8B illustrate examples of expandable fluidic channels and catheters in accordance with at least one example of the present disclosure.

FIG. 9 illustrates a method of use of an endoscope in accordance with at least one example of the present disclosure.

### SUMMARY

A multi-channel endoscope or ureteroscope can have a control section and an insertion section. The insertion section can have an insertion end with multiple openings where the multiple openings can correspond to at least one of a working channel, optical systems, fluid systems (such as air systems or water systems), or suction and removal systems. The multi-channel endoscope can include a user control mechanism and/or an actuator. In an example, the user control mechanism can operate the insertion section so it can articulate to reach a target location. A need has been identified to have a suction mechanism in at least one of the channels of the multi-channel endoscope such as a first suction channel which can be operated simultaneously or concurrently while other medical instruments are operated from within at least one separate working channel. A suction catheter can be disposed within a working channel and at least one medical instrument can be operated in a separate working channel. The suction catheter can be integrated with the insertion end and can extend and retract from an originating proximal position at an insertion end of an insertion section of the endoscope. A vacuum pressure source can be coupled to a proximal end of the suction channel proximate to the control section. The user control mechanism can control the position of the suction catheter and also simultaneously or sequentially activate the vacuum pressure source so the vacuum may be applied to a target site. In an embodiment, the fluid systems deliver irrigation to the surgical site via an irrigation channel. The irrigation channel may be the same or different from the working channel used for providing suction or the working channel which provides an area for the medical instrument(s) to be inserted therein. Additionally or alternatively, an irrigation catheter may be included in an irrigation channel; the irrigation catheter can be integrated with the insertion end and can extend and retract from an originating proximal position at an insertion end of an insertion section of the endoscope.

### DETAILED DESCRIPTION

An endoscope can be used to help facilitate access, visualization, or for manipulation of instrumentation during a procedure, such as which can be used to break up and remove one or more stones from the urinary tract. The endoscope can be at least partially inserted into an opening of a body of a patient or subject, such as for a urological procedure. In various examples, the endoscope can be partially inserted into any tract within a human body, such as to help remove small pieces of biological material.

For example, an endoscope can be used for visualization of an internal area of a tract within a patient, such as at an internal location where a procedure can be performed. The endoscope can include or be coupled to a camera component. The camera can transmit video or still pictures to a display screen locally or remotely located external to the patient. The endoscope can include or be coupled to a light source component, such as to provide light (e.g., via an optical fiber) at the internal location of the body for the camera to transmit visible images of the internal location. The endoscope can also have one or more channels or passages, e.g., working channels, through which one or more other instruments can pass.

For example, the endoscope can be a multi-channel endoscope. The multi-channel endoscope can include one or more working channels. Such a working channel can enable a user to pass and/or use one or more instruments during a procedure (such as one or more of a guidewire, a dilator, a basket, a laser fiber, a lithotripter, or one or more other instruments). For endoscopy in the urinary tract, the endoscope can be a ureteroscope. The ureteroscope can be flexible or otherwise specialized for use in the upper urinary tract. A challenge of using such a multi-channel ureteroscope or other endoscope is it can lack concurrent, simultaneous, or real-time suction or irrigation during certain urological procedures.

The present inventors have recognized, among other things, that an endoscope enabling concurrent or real-time fluidic action, such as suction or irrigation, during a urological procedure can be used such as to help remove stones or stone fragments, dust, or other target bodies. Devices and systems disclosed in this document can help reduce a need to break the stones into small enough fragments during the procedure such as to permit natural expulsion from the body—which can be a challenge of using an endoscope without integrated or suction capabilities. The devices and systems disclosed herein can help ease and speed the procedure such that a patient is substantially unencumbered of stones immediately following the procedure.

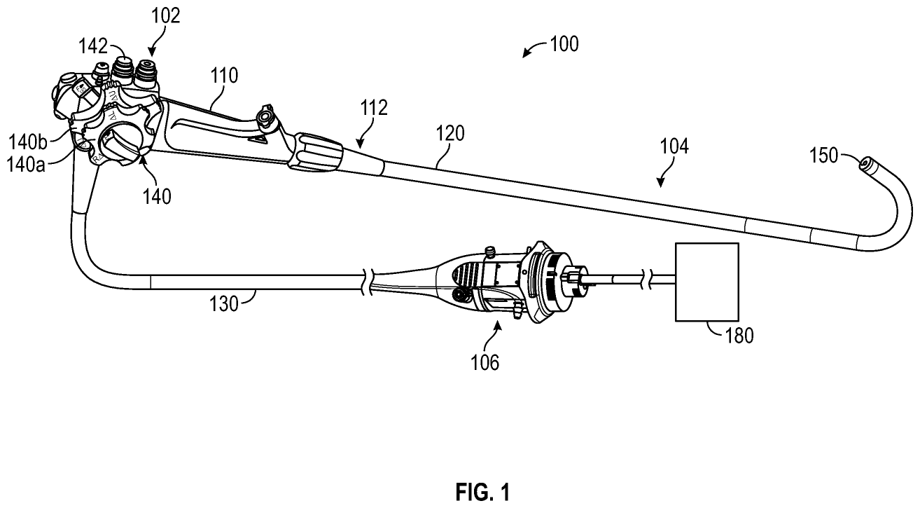

FIG. 1 illustrates an example of a multi-channel endoscope 100 (“endoscope”) such as a ureteroscope that can be used for urological treatments. The endoscope 100 can include a control section 102, an insertion section 104, and a connector section 106.

The control section 102 can be an intermediary section between the connector section 106 and the insertion section 104. The control section 102 can be the primary user interface of the endoscope 100. The control section 102 can include at least one component for operating components within or associated with the endoscope 100, such as a user control mechanism 140 or an actuator 142.

The user control mechanism 140 can be used to guide or control mechanical movements of the insertion section 104 of the endoscope 100. The user control mechanism 140 can include at least one of a button, lever, joystick, knob, dial, touch sensor, or the like. There can be more than one user control mechanism 140 included in or coupled to the control section 102. The control section 102 can also include a handle 110, or grip. A medical professional or other user can hold the handle 110 with one hand while operating the user control mechanism 140 with the other hand.

The actuator 142 can be included with or coupled to the control section 102 at a location remote from or proximate to the user control mechanism 140. The actuator 142 can include at least one actuator, such as one or more of a button, lever, joystick, knob, dial, touch sensor, or the like. The actuator 142 can control one or more mechanical or electrical components, such as those mechanical or electrical components that can be located within or associated with the control section 102 and can extend through the insertion section 104. In an example, there can be more than one actuator. For example, a first actuator can be couplable with a medical instrument and a second actuator can be couplable with a suction component.

The insertion section 104 can house mechanical or electrical components which can extend from at least the control section 102 through a lumen 120 of the insertion section 104. The lumen 120 can terminate at an insertion end 150. The insertion end 150 can have at least one openings corresponding to at least one channel.

The mechanical or electrical components within the endoscope 100 can also be housed within and extend through a universal cord 130. The universal cord 130 can be on an opposite end, or remotely located, from the insertion section 104. The universal cord 130 can couple any of a plurality of external systems 180 to the insertion section 104, as will be discussed further below.

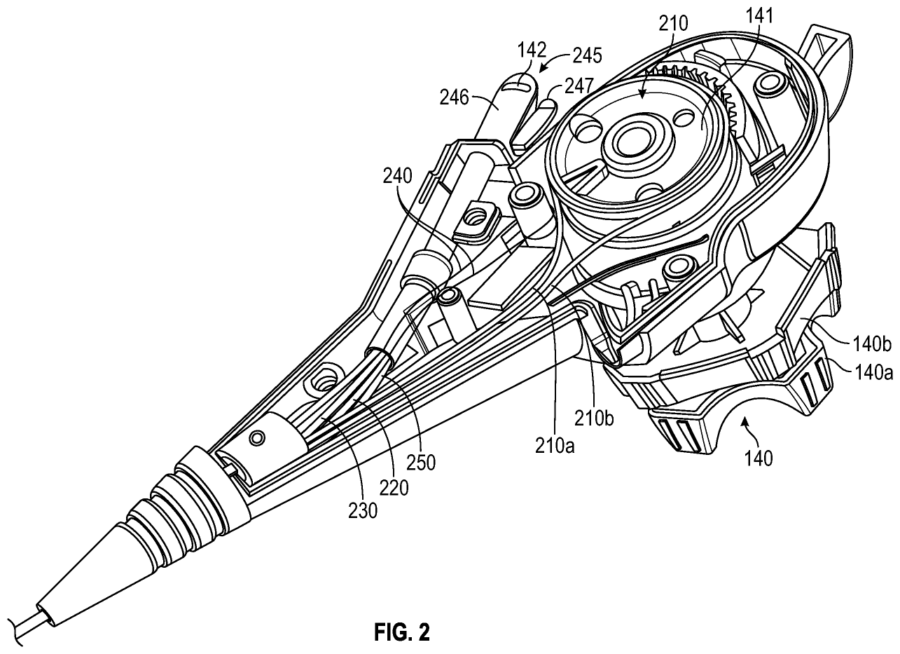

FIG. 2 illustrates a view of the internal components of the control section 102 of the endoscope 100. The control section 102 can include one or more electronic components 240 such as wires, electronic cables such as fiber optic cables, electronic circuitry chips, computer hardware, or the like. In an example, the one or more electronic components 240 can transmit one or more wired or wireless electrical signals, such as to or from the insertion end 150 of the insertion section 104 and from or to the connector section 106 or the control section 102, such as discussed further below. For example, the one or more electronic components 240 can communicate one or more imaging signals or light signals to or from at least one component at the insertion end 150.

In an example, the control section 102 can also include one or more operation connections 245. The one or more operations connections 245 can connect at least one suction component 246 or at least one fluid transfer component 247, such as via a lumen 120 in the insertion section 104 that is in fluid communication with the insertion end 150. The one or more operation connections 245 can further connect the at least one suction component 246, such as a suction valve, or the at least one fluid transfer component 247 (such as an air source line or an irrigation fluid supply line) with the connector section 106. The suction channel 250 and/or the at least one fluid transfer component 247 can include respective tubes that can allow fluid, such as air or liquid, or small solid particles such as debris, or biological remains acquired from within the patient, to pass through.

The control section 102 can also include at least one working channel 220 through which a surgical tool or other implement can be passed. For example, the at least one working channel 220 can provide a guidewire lumen passageway that is sized for permitting a guidewire 230 to pass. The guidewire 230 can be used to help provide for better navigation of the lumen 120 toward, to, or within the target location. The guidewire 230 can also allow for advancement of one or more of a dilator, a stent, a catheter, a target object (e.g., stone) removal implement, a drainage implement, or the like. The at least one suction component 246 can pass through the suction channel 250. In an example, the at least one suction component 246 can include a suction catheter 255, as described later.

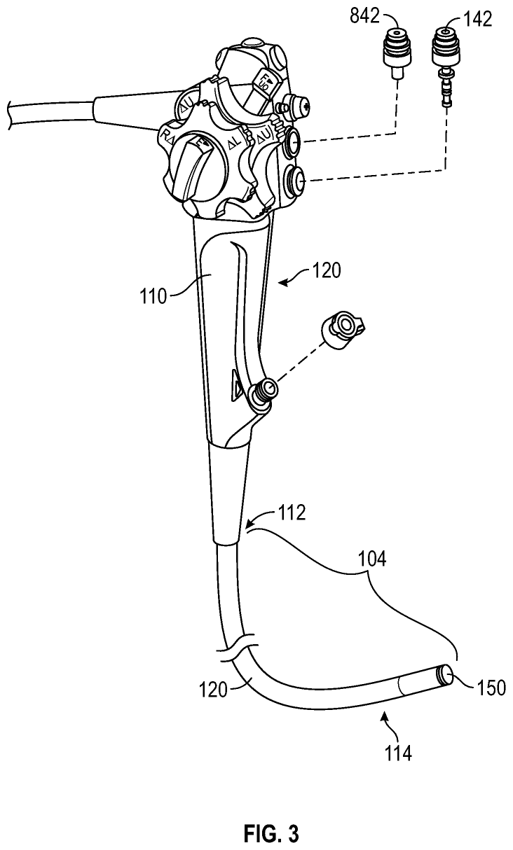

FIG. 3 shows an example of the control section 102 and the insertion section 104. The insertion section 104 can include the lumen 120. The insertion section 104 and the associated lumen 120 can be located at or proximate to a distal end 112 of the handle 110 of the control section 102 and coupled with a proximal end 114 of the insertion end 150. The insertion section 104 providing the lumen 120 can include a medical grade tube that can be sized and shaped to traverse one or more passages within the patient to a target location, such as within the urological anatomy of a patient. The insertion section 104 providing the lumen 120 can be made from a flexible material. This can allow the insertion section 104 providing the lumen 120 to more easily traverse passages within the body.

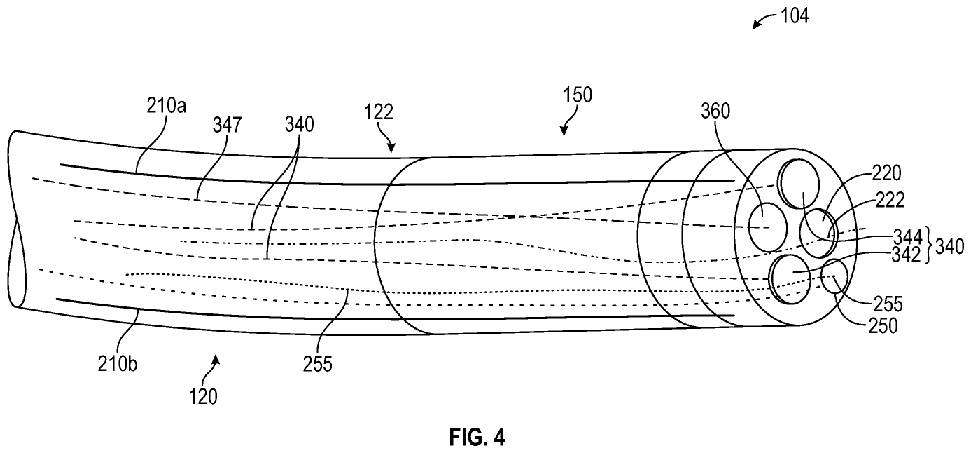

FIG. 4 shows an example in which the insertion end 150 can be located at the distal end 122 of the insertion section 104. The insertion end 150 can be sized and shaped to fit within a desired passage in the body, such as to be operable at and proximate to the target location. The insertion end 150 can include at least one of the following components: at least one optical component 340 such as at least one optical fiber bundle or other light guide 342, at least one video or photographic component 344 or optical coupler thereto, at least one fluid outlet 360, and at least one working channel 220, such as discussed in more detail below.

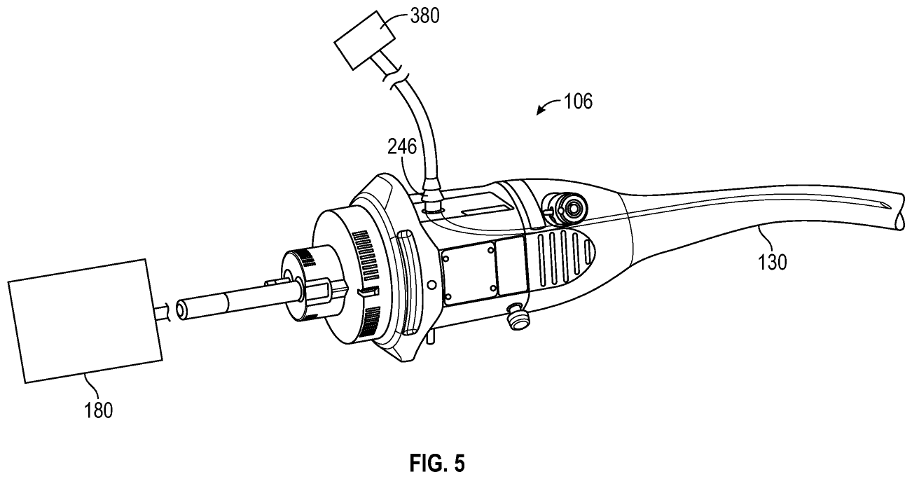

FIG. 5 shows an example in which the connector section 106 can connect the endoscope 100 with at least one external component or system 180. The connector section 106 can act as an interface conduit for the endoscope 100 to connect to one or more external components or systems 180. A universal cord 130 can be sized such that wires, cables, or tubes can pass through or traverse from the connector section 106 to the control section 102. The at least one external component or system 180 can include a light source, a video or photo interface or system, a fluid supply (such as an air supply or a water supply), a suction system, or the like.

For endoscopy in a urinary tract, as illustrated in FIG. 4, the flexibility of the lumen 120 can allow the lumen 120 to articulate or move within the tract. The lumen 120 can articulate upward, downward, right, left, or a combination of directions such as to permit the insertion end 150 to move or be placed at a target location. The lumen 120 of the insertion section 104 can include at least one mechanical system 210 such as a linkage member that can couple the user control mechanism 140 with a distal end 122 of the lumen 120. For example, a linkage member 210 can include one or more cables, wires, or other linkage members, such as can be associated with at least one rotating reel (or rotatable member) 141 internal the handle 110, as illustrated in FIG. 2. The at least one rotating reel 141 can maneuver, for example, the linkage member 210 to controllably couple the user control mechanism 140 and the distal end 122 of the lumen 120. For example, at least one mechanical system can be a first cable 210a can articulate the lumen 120 in a vertical direction. A second cable 210b can articulate the lumen 120 in a horizontal direction.

The first cable 210a can be connected with a first user control mechanism 140a which can be disposed externally on the handle 110. The second cable 210b can be connected with a second user control mechanism 140b which can be disposed externally on the handle 110. The first user control mechanism 140a and the second user control mechanism 140b can include respective rotatable components such as knobs, gears, handles, or the like. The first user control mechanism 140a can be stacked or share a common axis of rotation with the second user control mechanism 140b. In another example, the first user control mechanism 140a can be separately located from the second user control mechanism 140b. The first user control mechanism 140a and the second user control mechanism 140b can be coupled to any electromechanical system or other mechanism that can cause articulation of the insertion section 104 providing the lumen 120.

Within the lumen 120 extending along the insertion section 104 there can be one or more components or systems that can extend from the control section 102 to the distal end 122 or the insertion end 150. The insertion end 150 of the lumen 120 can house one or more individual components that can communicate with the one or more systems or components within the control section 102 or the connector section 106. Systems within the lumen 120 can include a fluid system 347 (such as an air or irrigation fluid delivery system), the suction channel 250, the at least one mechanical system 210 (such as to articulate the insertion section 104 providing the lumen 120), or at least one optical component 340 (such as a camera, a light source, fiber bundle or other illumination component).

The insertion section 104 can include at least one working channel 220. The at least one working channel 220 can be used to pass and use one or more instruments during a procedure (such as a guidewire, a dilator, a basket, a laser fiber, a lithotripter, or a combination of these or one or more other instruments). The at least one working channel 220 can be separate from the fluid system 347, the suction channel 250, the at least one mechanical system 210, and the at least one optical component 340. The instruments that can pass through the at least one working channel 220 can include one or more instruments that can be operated sequentially or substantially simultaneously, concurrently or separately. For example, a laser fiber 222 can be introduced through at least one working channel 220 such that the laser fiber 222 can be used to deliver laser energy pulses to help break down one or more desired target objects.

The suction channel 250 can carry a catheter 255, such as a suction catheter. The suction channel 250 can house or contain the suction catheter 255 such that the suction catheter 255 can be introduced via the suction channel 250. The suction catheter 255 can be integrated with the suction channel 250.

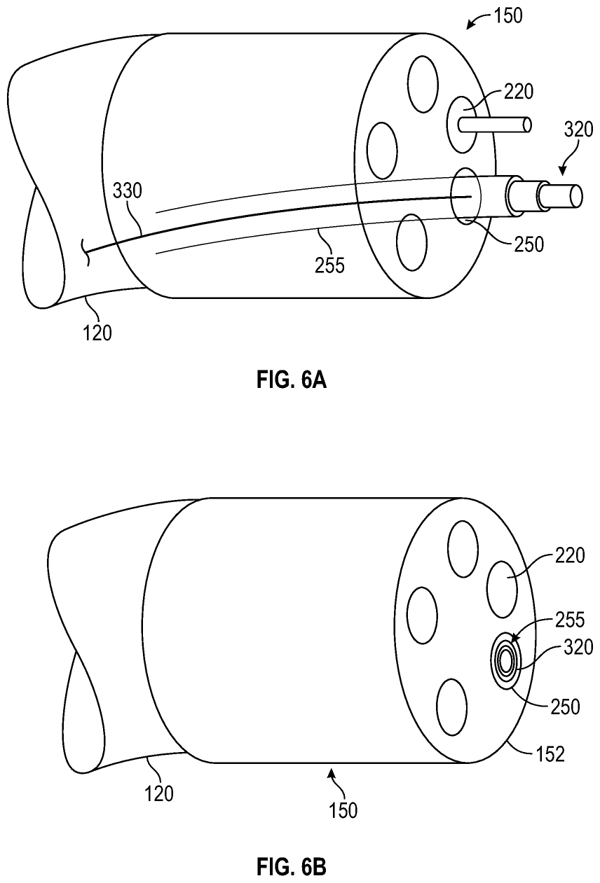

FIGS. 6A and 6B show an example in which the suction catheter 255 can include an extendible portion 320. The extendible portion 320 can be selectively operated to extend or retract from the suction channel 250. As illustrated in FIG. 6B, the suction catheter 255 is illustrated in a retracted state, such that it can be housed or contained within the insertion end 150. In the retracted state, an end of the suction catheter 255 can be in a position that the suction catheter 255 does not protrude, or only minimally protrudes, beyond the insertion end 150.

The suction catheter 255 can be fully or partially extendible. In a partially extendible configuration, as illustrated in FIG. 6A, at least a portion of the extendible portion 320 of the suction catheter 255 can extend beyond the insertion end 150. In a fully extendible configuration, the extendible portion 320 of the suction catheter 255 is extended completely such that substantially all of the extendible portion can extend beyond the insertion end 150.

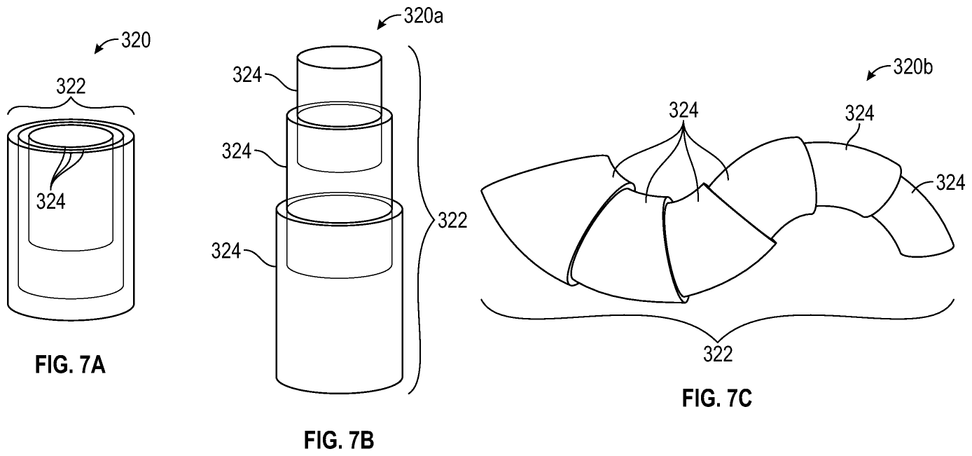

The extendible portion 320 can extend substantially linearly 320a, as illustrated in FIG. 7B or in an arcuate manner 320b, such as illustrated FIG. 7C. The extendible portion 320 can be retracted such that the extendible portion 320 can be housed within the lumen 120 when not deployed, such as illustrated in FIG. 6B. The extendible portion 320 can be housed or contained within the insertion end 150 when the extendible portion 320 is not in use. The extendible portion 320 of the suction catheter 255 can be completely or mostly within the insertion end 150 proximal of the distal-most portion of the insertion end 150. For example, when the extendible portion 320 is in use, at least a portion of the suction catheter 255 can be extended from the distal-most end of the insertion end 150 and toward or to the target location.

FIGS. 7A, 7B and 7C show examples in which the extendible portion 320 can include a shape memory extendible tube. The extendible portion 320 can be made from a plurality of shaft elements 324 that can be individually or collectively connected to the actuator 142, or any other component which can cause the extendible portion to extend and articulate. The extendible portion 320 can include or be made from concentric tubes, such as to form a telescoping portion 322. The actuator 142 can be operated to both extend or retract the telescoping portion 322, such as by movably positioning any or all of the plurality of shaft elements 324 distally outward or proximally inward. The actuator 142 can include or use a cable, wire, an electromechanical element, pneumatic or hydraulic elements such as a solenoid, or a motor or micromotor connected or coupled to at least one of the plurality of shaft elements 324.

In an example, a linkage can couple the actuator to an inner-most of a series of the concentric telescoping portion 322 which, at a proximal end, can include an outward radial flange. The outward radial flange can engage or couple with an inward radial flange of the next outward concentric shaft element. This arrangement can be repeated through each of the connected shaft elements 324 so a single or shared linkage can be used to form the telescoping portion 322.

In an example, each of the connected shaft elements 324 can form a seal, or have a seal, between the adjacent, or next, shaft element 324 in the telescoping portion 322. Each of the connected shaft elements 324 can also have a material disposed, formed, affixed or the like, to at least of one of a proximal or distal end of a shaft element 324 which can assist in forming a seal between the adjacent or next shaft element 324. The seal can assist in the next outer shaft element 324 preservation of the suction and can inhibit escape or release of any applied vacuum from between the shaft elements 324.

The extendible portion 320 can be coupled with the user control mechanism 140. In another example, the extendible portion 320 can be coupled with the actuator 142. The actuator 142 can be used to move the extendible portion 320 from a retracted position originating within the distal end 152 of the insertion end 150 to an extended position exposed outside of the distal end 152 and from the extended position to the retracted position. At least one cable 330 or wire or other mechanical or other linkage can be coupled with the actuator 142 and the extendible portion 320. The at least one cable 330 can control the position of the extendible portion 320, such as to position at or move between a retracted or extended position or to articulate the extendible portion 320. The at least one cable 330 can apply tension or a tensile force to the lumen 120.

The extendible portion 320 can include or be made from a shape memory material such as a shape memory alloy or a shape memory polymer. A hybrid of shape memory alloy and polymer can be used if desired. Examples of shape memory alloys can include copper-based alloys, iron-based alloys, or nickel-based alloys. Examples of shape memory polymers can include polyurethane based materials. In an example, Nitinol, a nickel-based shape memory alloy can be used.

The actuator 142 can operate at least one of articulation of the suction catheter 255, extension and retraction of the suction catheter 255, or activating or deactivating a vacuum pressure source 380. The actuator 142 can include a button, lever, joystick, knob, dial or touch sensor, or any other mechanical or electrical device that can transmit an operating control force or signal to the suction catheter 255. In an example, the actuator 142 can include a remote component that is not directly connected with the control section 102. For example, the actuator 142 can have components on remote, but electronically connected, computer systems. The actuator 142 can control the position of at least a portion of the suction catheter 255 such that the distal or other desired portion of the suction catheter 255 can be moved toward or to the target site or area at which suction is to be applied to remove material from the target site and from the patient. In an example, the actuator 142 can extend at least a distal portion of the suction catheter 255. In an example, the actuator 142 can extend the shaft elements 324 such that the shaft elements 324 of the suction catheter 255 extend past the insertion end 150. The actuator 142 can communicate signals or mechanically retract the shaft elements 324 such that the shaft elements 324 are retracted into and housed within the suction channel 250. For example, the actuator 142 can be coupled with a cable, wire, an electromechanical element, pneumatic or hydraulic elements such as a solenoid, or a motor or micromotor. In another example, the actuator 142 can deactivate the vacuum pressure source 380 to remove the material from the patient.

The actuator 142 can operate at least one operation of the suction catheter 255. The actuator 142 can extend or retract the suction catheter 255. The actuator 142 can activate or deactivate a vacuum pressure source 380. The actuator 142 can control the articulation of the suction catheter 255. In an example, the actuator 142 can have separate components each of which controls one of articulation of the suction catheter 255, extension and retraction of the suction catheter 255, or activating or deactivating a vacuum pressure source 380. The actuator 142 can operate the suction valve or other component which can actuate a system to supply a vacuum flow through the suction catheter 255.

In an example, the proximal end of the suction channel 250 can be coupled with the vacuum pressure source 380 within the control section 102, within the proximal portion 121 of the lumen 120, or at any location within or remote to the endoscope 100. The vacuum pressure source 380 can be coupled with the connector section 106. The universal cord 130 can couple the vacuum pressure source with the control section 102 and the suction channel 250. The suction catheter 255 can be disposed within the suction channel 250 such that the vacuum pressure can be transmitted to and through the suction catheter 255.

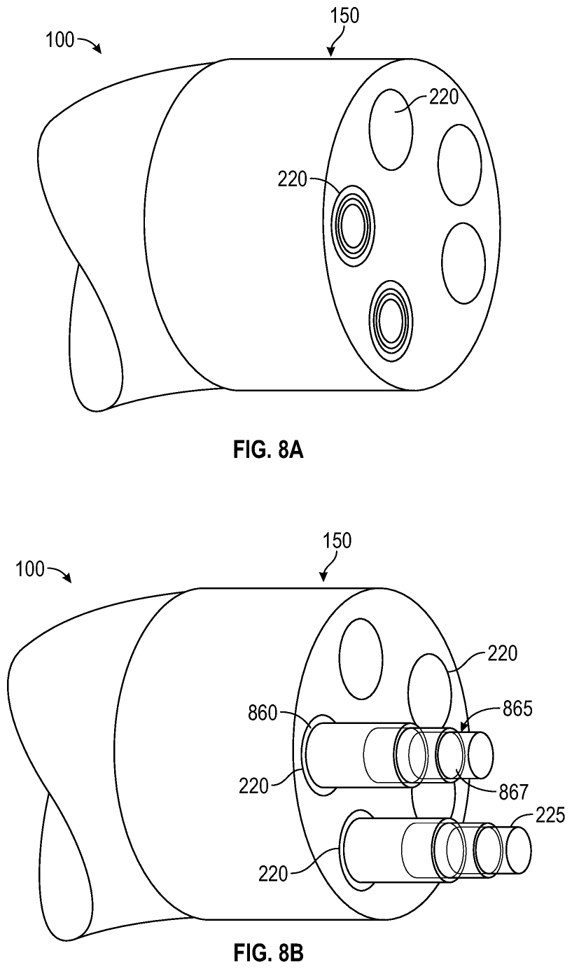

FIGS. 8A and 8B illustrate an example of an insertion end 150 of a multi-channel endoscope 100 in which one of the working channels 220 can be a fluidic channel 860. The fluidic channel 860 can be a channel configured to receive or house a fluidic catheter 865 such as an irrigation catheter or a suction catheter. The fluidic catheter 865 can apply a fluid, such as a low-pressure vacuum or an irrigation liquid (e.g. water, saline, medication, or the like) to the target site. The fluidic channel 860 can be a separate channel from the previously discussed suction channel and can be configured to receive or house the fluidic catheter 865 as an irrigation catheter 867. In an example in which the fluidic channel 860 includes the irrigation catheter 867, the irrigation catheter 867 can articulate and extend similar to the suction catheter 255 discussed above. The irrigation catheter 867 can be controlled by an irrigation actuator 842, which can be the same actuator as an actuator for a suction catheter or it can be a separate actuator. In an example in which the irrigation actuator 842 illustrated in FIG. 3 is a separate actuator, the irrigation actuator 842 can control the extension of the irrigation catheter 867 from an originating position, as illustrated in FIG. 8A, to an extended (partially extended or fully extended) position, as illustrated in FIG. 8B. The irrigation catheter 867 in an originating position can be oriented so it does not protrude or extend past the end of the insertion end 150. The irrigation catheter 867, in an extended position, can extend to the target site. The irrigation catheter 867 can operate concurrently, simultaneously, or separately from a suction catheter or other components extending within the multi-channel endoscope.

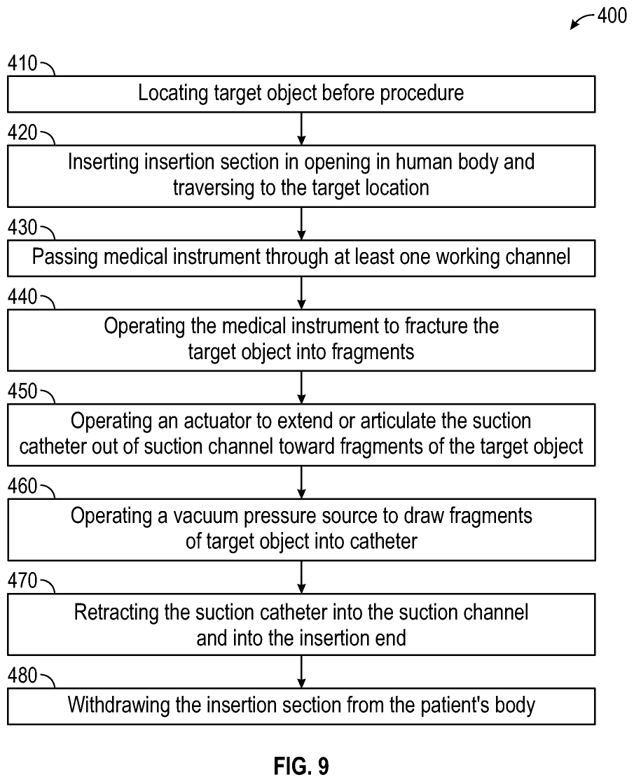

As illustrated and described in FIG. 9, the method 400, below, the catheter 255, such as a fluidic catheter, suction catheter, or irrigation catheter, can be a component within the multi-channel endoscope and can be used to assist in the removal of one or more target objects from a patient's body. The target objects to be removed can include fragments of stones or calculus within a kidney or the urinary tract.

At 410, at least one target object can be identified to be removed from a body with a multi-channel endoscope. A medical professional or other user can use the endoscope or another instrument to identify the location of the target object to be removed. Such methods can be by ultrasound, x-ray, or the like. After locating the target object, the following acts can be performed to remove the target object and to treat the patient.

At 420, the insertion section 104 can be inserted into an opening in a human body, such as an incision or the urethra. The insertion section 104 can pass through the bladder and ureter to the kidney or other target location. The insertion section 104 can include multiple channels, such as multiple working channels.

At 430, upon approaching the target location, the at least one working channel can be configured to accommodate or receive at least one medical instrument. The at least one medical instrument can pass from the control section, or handle, to the distal end of the insertion section 104. At least one of working channel, or a separate working channel can be configured to be a suction channel that can house a suction catheter. The suction catheter can extend from the control section, or handle, to a distal end of the insertion section. A medical instrument can be passed through the at least one working channel. The medical instrument can be selected for use to treat the target object.

At 440, a treatment actuator can be used to treat the target object by fracturing or breaking the target object into smaller pieces. The smaller pieces can be sized to fit within and removed by the suction catheter. In an example, a laser fiber can be passed through the at least one working channel and used to deliver one or more laser pulses to help break down the target object.

At 450, the actuator can be operated to extend, or articulate, the suction catheter away from the distal end of the insertion end. The user can operate a second actuator to extend or articulate the suction catheter away from the originating location within the distal end of the insertion end. The user can extend the suction catheter until a distal end of the suction catheter can be placed proximate to the target object. The user can operate the actuator such that the suction catheter can be extended from the insertion end to the target location. The actuator can extend individual sections of telescoping portions of the suction catheter. The actuator can extend multiple or all of the telescoping sections sequentially, concurrently, or simultaneously.

The user can operate the actuator to articulate the suction catheter. The user can also operate a second actuator to articulate the suction catheter. The user can operate the actuator or the second actuator to move the suction catheter upward, downward, right, left or a combination thereof.

At 460, the user can then operate the vacuum pressure source to draw the fragments of the target object into the suction catheter. The suction catheter can be operated independently, sequentially or substantially simultaneously with the medical instrument. For example, breaking up the target object can be performed independently of drawing the fragments of the target object into the suction catheter. In this example, the user can use the suction catheter during a procedure without the user needing to remove a medical instrument from the at least one working channel or without needing to remove the endoscope from the patient's body.

At 470, after drawing in or removing substantially all, or all, of the target object fragments the user can withdraw or retract the suction catheter into the suction channel of the insertion end of the insertion section. The user can then pass any further medical instruments through the at least one working channel, such as a separate working channel from the suction channel, to take any further steps in the medical procedure.

At 480, the user can withdraw the insertion section from the patient's body.

The above description includes references to the accompanying drawings, which form a part of the detailed description. The drawings show, by way of illustration, specific embodiments in which the invention can be practiced. These embodiments are also referred to herein as “aspects” or “examples.” Such aspects or example can include elements in addition to those shown or described. However, the present inventors also contemplate aspects or examples in which only those elements shown or described are provided. Moreover, the present inventors also contemplate aspects or examples using any combination or permutation of those elements shown or described (or one or more features thereof), either with respect to a particular aspects or examples (or one or more features thereof), or with respect to other Aspects (or one or more features thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

The above description is intended to be illustrative, and not restrictive. For example, the above-described aspects or examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as aspects, examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A multi-channel endoscope system having an endoscope device with control section and an insertion end, and a vacuum pressure source coupled with the endoscope device, the multi-channel endoscope system comprising:
the endoscope device, comprising:
an actuator coupled with the control section;
at least one working channel; and
a fluidic channel including:
a catheter configured to extend from an originating position within the insertion end of the multi-channel endoscope system and retract within the insertion end;
wherein the actuator controls a position of the catheter and operates the catheter.

2. The multi-channel endoscope system of claim 1, further including at least one guidewire sized and shaped for being received in the at least one working channel.

3. The multi-channel endoscope system of claim 1, further including a laser fiber.

4. The multi-channel endoscope system of claim 1, further comprising:
a user control mechanism that positions a lumen that extends from a distal end of the control section to a proximal end of the insertion end;
wherein the lumen houses the at least one working channel and the fluidic channel.

5. The multi-channel endoscope system of claim 1, wherein the catheter is a suction catheter including a telescoping portion with a plurality of shaft elements.

6. The multi-channel endoscope system of claim 5, wherein the actuator extends and retracts the telescoping portion.

7. The multi-channel endoscope system of claim 1, wherein the actuator comprises at least one of a button, a lever, a joystick, a knob, a dial, or a touch sensor.

8. The multi-channel endoscope system of claim 1, wherein the catheter of the fluidic channel is at least one of a suction catheter or an irrigation catheter.

9. A multi-channel ureteroscope having a control section with a handle, a lumen with a distal insertion end and a proximal controlling end, the multi-channel ureteroscope comprising:
a user control mechanism coupled to the handle;
at least one working channel traversing an internal portion of the handle and extending to the distal insertion end for receiving at least one medical instrument; and
a suction channel traversing the internal portion of the handle and extending to the distal insertion end;
wherein, the suction channel is configured to receive a suction catheter that extends through the suction channel and to a treatment end.

10. The multi-channel ureteroscope of claim 9, wherein the user control mechanism is configured to manipulate at least one of the at least one medical instrument or the suction catheter.

11. The multi-channel ureteroscope of claim 9, wherein the suction catheter includes an extendible portion.

12. The multi-channel ureteroscope of claim 11, wherein the extendible portion includes a telescoping portion with a plurality of shaft elements.

13. The multi-channel ureteroscope of claim 9, wherein the handle includes a first actuator couplable to the at least one medical instrument.

14. The multi-channel ureteroscope of claim 13, wherein the handle includes a second actuator couplable to the suction catheter for extending and retracting the suction catheter.

15. The multi-channel ureteroscope of claim 9, wherein the suction catheter is configured to be coupled to a remote device for extending and retracting the suction catheter.

16. A method of removing a target object from a body using an endoscope including a working channel and a fluidic channel the method comprising:
fracturing the target object into fragments;
wherein the working channel is configured to accommodate a medical instrument to fracture the target object;
extending a fluidic catheter from an original retracted configuration in the fluidic channel towards the fragments; and
wherein in the original retracted configuration the fluidic catheter is housed within the fluidic channel;
applying at least one of suction or irrigation through the fluidic catheter toward at least a portion of the target object.

17. The method of claim 16, wherein extending the fluidic catheter is a suction catheter and includes extending a telescoping portion of the suction catheter to the target object.

18. The method of claim 17, further comprising:
articulating the telescoping portion to the target object.

19. The method of claim 16, further comprising:
inserting an insertion section of the endoscope in a patient towards the target object; and
passing the medical instrument through the working channel to the target object;
wherein the medical instrument includes a laser fiber.

20. The method of claim 16, wherein fracturing the target object with the medical instrument and applying at least one of suction or irrigation are independently actuatable via a first actuator and a second actuator.
